(19) United States
(12) Patent Application Publication
(10) Pub. No.: US 2020/0008714 A1
(43) Pub. Date: Jan. 9, 2020

(54) METHOD AND APPARATUS FOR HUMAN FALL DETECTION WITH POWER-SAVING FEATURE

(71) Applicant: HONEYWLD TECHNOLOGY CORP., Hsinchu City (TW)

(72) Inventor: JEN-CHIEN HSU, HSINCHU CITY (TW)

(21) Appl. No.: 16/028,086

(22) Filed: Jul. 5, 2018

(51) Int. Cl. A61B 5/11; G08B 21/04

(52) U.S. Cl. CPC A61B 5/1117; A61B 2503/08; G08B 21/0446

(57) ABSTRACT

The disclosure is related to a method and an apparatus for human fall detection with power-saving feature. In the method, a processor is set in a sleep state under a normal operating condition, and sensor data generated by a sensor unit of the apparatus worn on a person is stored in a buffer. A processor of the apparatus is woken up from a sleep state when the processor receives a collision signal, and the processor then retrieves the buffered sensor data and current sensor data. The sensor data is processed by a fall detection program. The apparatus will generate an alarm for a fall event if the sensor data meets the preset fall conditions; otherwise, the processor returns to the sleep state. The apparatus saves power by avoiding huge data calculation while staying in the sleep state until being woken up by the collision signal.

### BACKGROUND OF THE INVENTION

### 1. Field of the Invention

The disclosure is related to an apparatus for detecting a human's fall, and in particular to an apparatus for human fall detection with a power-saving feature, and a method for saving power implemented in the apparatus.

### 2. Description of Related Art

Human fall detection is one of the major issues in a healthcare system. The fall detection technology is a developing technology in the field of healthcare that can be implemented in a care system for the elderly, infirmed, or disabled population.

Conventional fall detection technology generally utilizes a portable device that is worn on the person under care of the care system. The portable device is such as a wristband or a necklace with sensors for monitoring fall actions of the person who is especially a member of the elderly population. In particular, in order to detect the fall, the portable device worn on the person under care is required to be in operation continuously for constantly issuing detection signals to the care system.

For example, in the conventional technology, the portable device, e.g. the wristband or the necklace, utilizes sensors such as an accelerometer and a gyroscope to detect a fall action when the person who wears the portable device falls. In general, the accelerometer measures a change of acceleration, e.g. a change of velocity toward the center of Earth due to the Earth's gravity. A gyroscope measures angular velocities and converts them to orientation. When the change of acceleration exceeds a threshold or orientation exceeds a threshold set by the care system, a fall signal will be generated.

Further, the accelerometer can also detect a collision event by recognizing a rapid negative acceleration of the portable device. By these features, the conventional care system can accurately recognize the fall action of the person under care. The system issues an alarm when it receives a fall signal, a collision signal and a final rest signal from the portable device in sequence.

For example, the conventional fall detection process is generally based on a change of an acceleration value with time. The acceleration value is computed from data generated by an accelerometer disposed in the device worn by the person to under care. Reference is made to FIG. 1, which shows a chart illustrating a trend of an acceleration value a(t) with time t. In the fall detection process, a fall event is detected as a fall state when the acceleration value a(t) approaches zero (free fall), a collision state when the acceleration value a(t) increases rapidly, and a rest state when the acceleration value a(t) is maintained at a stable value in the trend of the acceleration value a(t) with time t.

Such automatic fall detection technologies are well known in the art. To achieve the fall detection, the sensors disposed in the portable device worn on the person under care are required to operate continuously for collecting movement data, and a processor of the portable device is required to compute the data generated by the sensors continuously. Therefore, the portable device will excessively consume electric power and suffer from drawbacks such as unsustainable operation since the electricity of a battery of the device is easily exhausted.

### SUMMARY OF THE INVENTION

According to one aspect of the invention, there is provided a method and an apparatus for human fall detection with a power-saving feature. The apparatus for human fall detection is such as a portable device worn on a person, whom may be a member of the elderly, infirmed, or disabled population in need of a care system.

For the requirement of care, the apparatus is required to perform a full-time monitoring to the person under care, and the apparatus will continuously consume its electric power, e.g. the power supplied by a battery. Therefore, the apparatus for human fall detection with power-saving feature in accordance with the invention is provided. The apparatus includes a processor, a power management unit inside the processor, a sensor unit, a buffer, and a memory unit. The power management unit is used to set the processor to an awake state or a sleep state. The sensor unit can be an accelerometer or an accelerometer combined with other sensors such as a gyroscope operatively coupled with the processor and used to measure acceleration data correlated to the apparatus or acceleration data combined with other types of sensor data such as angular velocities. The buffer stores sensor data generated by the sensor unit when the processor is in the sleep state. The memory unit stores a fall detection program that is executed by the processor for performing a method for human fall detection.

In one aspect of the invention, the processor is set to be in the sleep state under a normal operating condition, and the sensor unit is continuously in operation for generating sensor data correlated to the apparatus, e.g. the acceleration data or the acceleration data combined with other types of sensor data such as angular velocities. The buffer keeps storing the sensor data when the processor is in the sleep state. The processor is woken up from the sleep state when it receives a collision signal from the sensor unit. The collision signal is generated by the accelerometer of the sensor unit when the accelerometer determines a collision event, e.g. an acceleration value generated by the sensor unit is larger than a first threshold.

When the processor is woken up from the sleep state, the processor then retrieves a latest previous sensor data stored in the buffer. A fall event is confirmed if the latest previous sensor data stored in the buffer and the current sensor data meet the fall conditions.

In another aspect of the invention, when the processor is woken up from the sleep state, the processor acquires current acceleration data generated by the sensor unit. If the current acceleration value calculated from the acceleration data is larger than a second threshold, the processor then retrieves a latest previous sensor data stored in the buffer; otherwise, the processor enters the sleep state.

It should be noted that the buffer can be a buffer inside the processor, a buffer inside the sensor unit, or an external memory. Further, as compared with the generated current sensor data, the sensor data stored in the buffer is generally the latest previous sensor data when the processor is in the sleep state.

By storing the sensor data in the buffer when the processor is in sleep state, setting up a condition to wake up a processor, and retrieving the sensor data in the buffer after the processor is woken up, the aforementioned aspects of the invention solves the problem of too much power consumption while performing the method for human fall detection because the apparatus is required to continuously operate for monitoring the person who wears the apparatus.

These and other advantages and aspects of the invention will become apparent to those skilled in the art upon a reading of the following detailed description of the invention, in conjunction with the accompanying drawings.

### BRIEF DESCRIPTION OF THE DRAWINGS

FIG. 1 shows a chart illustrating a trend of an acceleration value with time in a conventional fall detection process;

FIG. 2 shows a block diagram depicting the main circuits of the apparatus for human fall detection according to one embodiment of the disclosure;

FIG. 3 shows another block diagram depicting the main circuits of the apparatus for human fall detection according to another embodiment of the disclosure.

FIG. 4 shows a flow chart describing a process of generating a collision signal in the method for human fall detection in one embodiment of the disclosure;

FIG. 5 shows a flow chart describing a process of waking up a processor in the method for human fall detection in one further embodiment of the disclosure;

FIG. 6 shows a flow chart describing the method for human fall detection according to a first embodiment of the disclosure; and

FIG. 7 shows a flow chart describing the method for human fall detection according to a second embodiment of the disclosure.

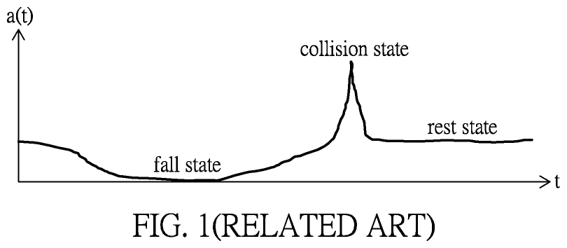

### DETAILED DESCRIPTION OF THE PREFERRED EMBODIMENTS

The present invention will now be described more fully with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The disclosure describes an apparatus and a method for human fall detection. The apparatus is such as a portable device provided for a person to conduct fall detection. The apparatus includes a sensor unit such as an accelerometer that is used to measure acceleration of the apparatus, or a gyroscope for measuring an orientation and angular velocities. For example, the sensor can be a three-axis accelerometer that is used to measure three acceleration component values in three axial directions, e.g. three acceleration vectors in X-axis, Y-axis and Z-axis directions. An acceleration value can be calculated according to the acceleration vectors. It is well known that a magnitude of the three axial vectors is equal to a square root of a sum of squares of each of the components.

However, for detecting human fall, the apparatus worn on the person under care should be in full-time operation, and the limitation of battery power will affect the long-term operation of the apparatus. To reduce power consumption of the apparatus, a power-saving scheme is applied to the apparatus for human fall detection in accordance with the invention. According to one aspect of the invention, the power-saving scheme applied to the apparatus allows a processor of the apparatus to be in a sleep state most of the time until it is aware of a collision event. When the processor is in the sleep state, the data generated by the sensor of the apparatus are stored in a buffer. The buffer is configured to store the latest or a few limited data within a short period of time, e.g. 1 second, due to a capacity limitation of the buffer.

To wake up the processor from the sleep state, a condition for re-activating the processor can be set. In an exemplary example, the processor is activated to be in an awake state when it receives a collision signal from the sensor unit. The collision signal is generated if the sensor unit senses a large change of the acceleration value. Therefore, a collision threshold will be introduced for determining if the large change qualifies as a collision event. In the meantime, the processor of the apparatus can be re-activated to be in the awake state for processing the data retrieved from the buffer.

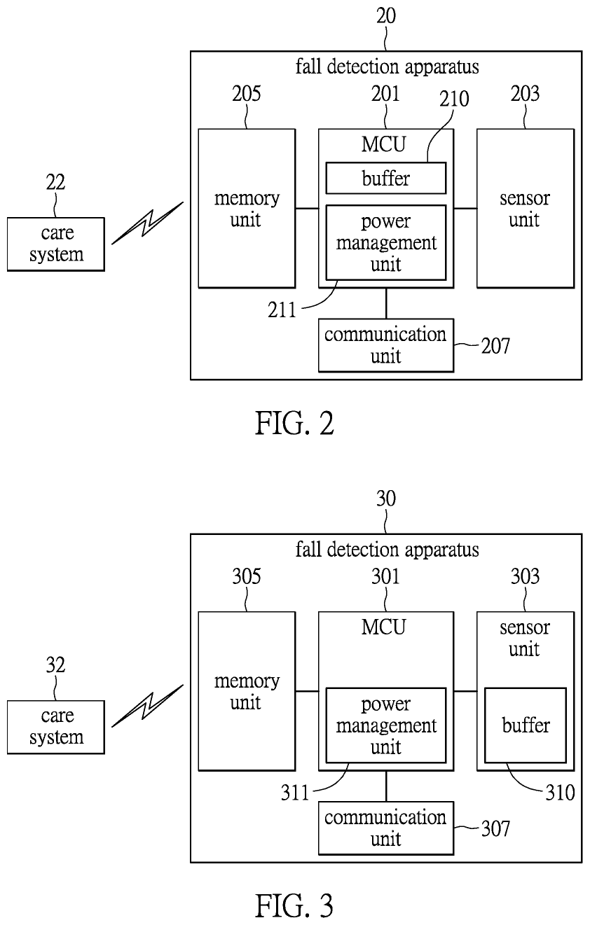

FIG. 2 shows a block diagram depicting the main circuits of the apparatus for human fall detection according to one embodiment of the disclosure.

A fall detection apparatus 20 is illustrated in the diagram. The apparatus 20 is such as a portable device worn on a person under care. For example, the portable device utilizes an inside sensor unit such as an accelerometer to sense the movement of the person. The portable device can be a wristband, necklace, or even a mobile phone executing a fall detection program that is required to be in full-time operation for performing fall detection.

This apparatus 20 includes a processor that can be a micro-processor, e.g. the shown MCU 201, for processing data generated by a sensor unit 203. The sensor unit 203 can be exemplified as an accelerometer that is operatively coupled with the MCU 201, and is used to measure acceleration data correlated to the apparatus 20. In an exemplary example, the accelerometer installed in the apparatus 20 is such as a three-axis accelerometer that is used to measure three acceleration component values in three axial directions. The acceleration value that is calculated according to the three acceleration components values refers to a square root of a sum of the squares of the three acceleration vectors.

In the present embodiment, the MCU 201 includes an internal buffer 210 that is used to store the sensor data generated by the sensor unit 203. A power management unit 211 is provided inside the MCU 201 for setting the processor to an awake state or a sleep state. It should be noted that, by the power management unit 211, the MCU 201 is set in the sleep state under a normal operating condition, and is woken up by the power management unit 211 when it meets the condition for re-activating the MCU 201.

The apparatus 20 includes a memory unit 205 that is operatively coupled with the MCU 201. The memory unit 205 generally acts as a system memory of the apparatus 20, and in particular, stores a fall detection program that is executed by the MCU 201 for performing the method for human fall detection. In one further embodiment of the disclosure, the apparatus 20 includes a communication unit 207 that is operatively coupled with the MCU 201 and is used to communicate with a care system 22. When the apparatus 20 detects a fall event by the method for human fall detection, an alarm is generated and transmitted to the care system 22 through the communication unit 207.

In addition to using the buffer 210 inside the processor to store the sensor data generated by the sensor unit 203, the buffer for storing the sensor data can also be a buffer inside the sensor unit 203. Reference is made to FIG. 3, showing the block diagram of the main circuits of the apparatus in another embodiment of the disclosure.

A fall detection apparatus 30 is provided. In the current embodiment, the apparatus 30 includes an MCU 301 for processing sensor data generated by a sensor unit 303. A power management unit 311 inside the MCU 301 is used to manage an operating state of the MCU 301. By the power management unit 311, the MCU 301 is set to a sleep state under a normal operating condition, and set to an awake state when it meets the condition for re-activating the MCU 301 from the sleep state. The apparatus 30 includes a memory unit 305 that acts as a system memory of the apparatus 30, and stores the fall detection program executed by the MCU 301 for performing the method for human fall detection. The apparatus 30 also includes a communication unit 307 for communicating with a care system 32.

According to the current embodiment, the sensor unit 303 includes an internal buffer 310 that is used to store the sensor data generated by the sensor unit 303. It should be noted that the buffer storing the sensor data can be the buffer (210, FIG. 2) inside the processor, the buffer (310, FIG. 3) inside the sensor, or an external memory.

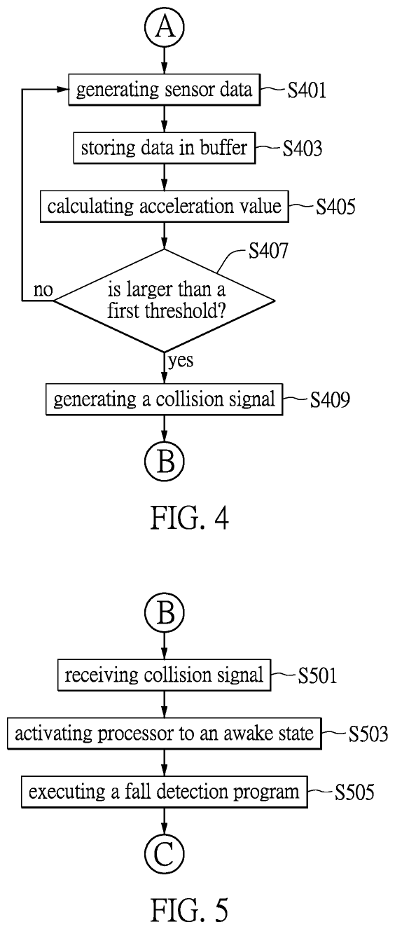

In the method for human fall detection in accordance with the invention, the processor of the apparatus is set to the sleep state in the normal operating condition and switched to the awake state when it meets a specific condition, e.g. receiving a collision signal. FIG. 4 shows a flow chart describing a process of generating the collision signal in the method according to one embodiment of the disclosure.

In this process (A), in step S401, the sensor unit of the apparatus worn on a person under care continuously generates sensor data, e.g. the acceleration values generated by the accelerometer or the acceleration values combined with the angular velocities generated by the gyroscope. In step S403, the sensor data is stored in a buffer inside the apparatus, or in an external memory. In step S405, the acceleration data is such as a raw data generated by the sensor unit and is provided to calculate an acceleration value for determining if any collision event occurs.

In step S407, in the sensor, it is determined that whether or not the acceleration value is larger than a first threshold. It should be noted that the first threshold is set by the system for determining if any collision event is detected. The collision signal is generated (step S409) by the sensor unit and the method proceeds to a process (B) described in FIG. 5 when the sensor unit determines that the acceleration value is larger than this first threshold. Otherwise, the process goes back to step S401 if the acceleration value is not larger than the first threshold.

FIG. 5 next shows a flow chart describing a process of waking up the processor in the method for human fall detection in one further embodiment of the disclosure.

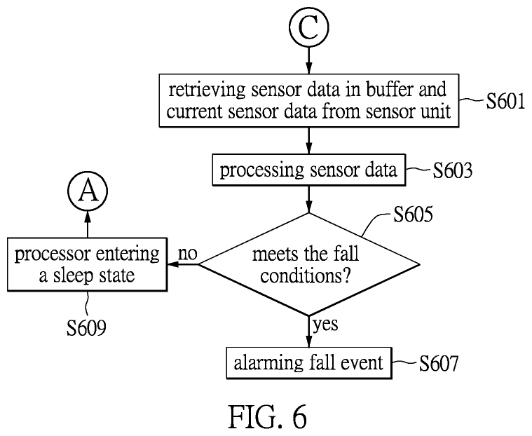

In step S501 of the process (B), the power management unit or any agent program running with lowest power in the processor receives the collision signal generated by the sensor in the process (A). In step S503, the processor is activated to be in the awake state for instantly executing a fall detection program (step S505). The next process (C) described in FIG. 6 is then performed.

FIG. 6 shows a flow chart describing the method for human fall detection in a first embodiment of the disclosure.

In step S601 of the process (C), the processor retrieves the sensor data stored in the buffer of the apparatus and the current sensor data from the sensor unit after the processor is set to the awake state when receiving the collision signal. In step S603, the processor processes the sensor data according to the fall detection program. It should be noted that, rather than the generated current sensor data, the sensor data stored in the buffer is the latest previous sensor data. Further, when the processor is in the sleep state, the sensor still operates and continuously generates the sensor data. It should also be noted that, while the sensor data is continuously generated, the buffer is configured to only store the latest or a few limited data and abandon the older data since the memory has a capacity limitation.

In step S605, the processor determines if the sensor data meets the fall conditions set by the fall detection program by comparing the sensor data with the fall conditions. It should be noted that this fall conditions set by the fall detection program is used to confirm a fall event detected by the apparatus. A fall event is confirmed and raises an alarm (step S607) when the sensor data meets the fall conditions; otherwise, the processor enters the sleep state (step S609) and goes back to process (A).

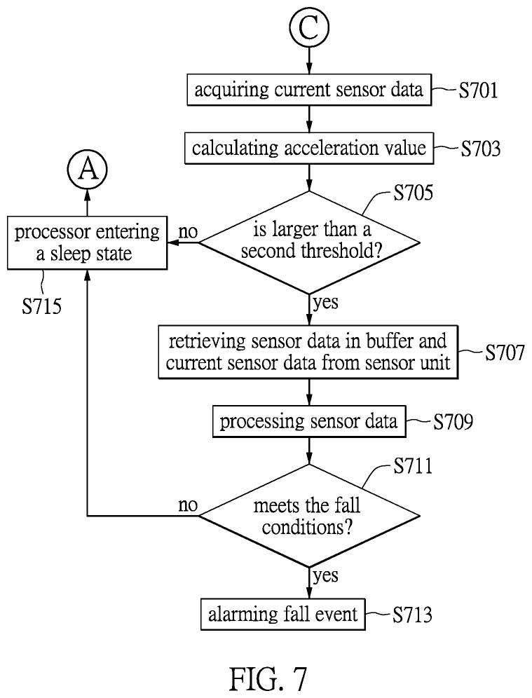

FIG. 7 shows one further embodiment showing a flow chart of the method for human fall detection of the disclosure. The above-mentioned process (A) discloses the sensor of the apparatus worn on a person under care, which continuously generates the sensor data when the processor is in the sleep state. The sensor data is then converted to the acceleration value for determining if any collision event occurs. When the sensor determines the acceleration value to be larger than the first threshold, the apparatus meets a collision event and generates a collision signal. Then, the process (B) describes the power management unit or any agent program running with lowest power for lowest operation of the apparatus in the processor receiving the collision signal, and the processor is activated to be in the awake state for instantly executing a fall detection program.

In step S701 of the process (C) according to the present embodiment, when the processor is woken up from the sleep state, the current acceleration data and this waking message will be transmitted to the power management unit or an agent program running with the lowest power in the processor. In step S703, when the processor acquires the current sensor data, an acceleration value is calculated. The processor in step S705 determines if the acceleration value is larger than a second threshold. It should be noted that, different from the first threshold in the above embodiment, the second threshold is introduced for the apparatus to more accurately detect the collision by using the processor to calculate the acceleration value.

If the acceleration value is found to be larger than the second threshold, in step S707, the processor is configured to retrieve the stored sensor data in the buffer, namely the latest previous sensor data, and the current sensor data from the sensor unit. Otherwise, when the acceleration value is not larger than the second threshold, in step S715, the processor enters the sleep state again.

Once the processor acquires the sensor data, in step 709, the sensor data is processed. In step S711, by comparing the sensor data with the fall conditions set by the fall detection program, it is determined that if a fall event occurs. The apparatus generates an alarm for a fall event if the sensor data meets the fall conditions (step S713). However, if the sensor data does not meet the fall conditions, the step S715 is performed and the method goes back to process (A). The processor will return to the sleep state.

In addition to providing the apparatus with the power-saving feature to monitor the person wearing the apparatus, the apparatus can also be applied to monitoring other people who are generally put in dangerous situations. For example, a police or a firefighter who are often faced with danger can utilize the apparatus to provide an instant alert when falling.

In sum, according to the above embodiments of the apparatus and method for human fall detection, the processor of the apparatus which is required to be in full-time operation is configured to be in the sleep state or in the awake state; for example, the processor can stay in the sleep state until it is woken up to perform the method only if it receives the collision signal generated by the sensor. Further, in the method, the apparatus will raise an alarm if it detects a fall event, but otherwise the processor will return to the sleep state. Therefore the apparatus can save much power because its processor can avoid huge data calculation by this mechanism.

It is intended that the specification and depicted embodiments be considered exemplary only, with a true scope of the invention being determined by the broad meaning of the following claims.

1. (canceled)

2. (canceled)

3. (canceled)

4. (canceled)

5. (canceled)

6. (canceled)

7. A method for human fall detection, comprising:
setting a processor to a sleep state;
storing sensor data generated by a sensor unit of an apparatus worn on a person under care in a buffer, wherein the sensor data is continuously generated and the buffer is configured to store the latest limited sensor data;
waking the processor up from the sleep state when receiving a collision signal from the sensor unit, wherein the collision signal is generated by an accelerometer when the accelerometer determines that an acceleration value calculated from current sensor data is larger than a first threshold;
the processor comparing current acceleration value calculated from the current sensor data with a second threshold;
the processor retrieving the latest previous sensor data stored in the buffer and the current sensor data from the sensor unit if the current acceleration value is larger than the second threshold; otherwise, the processor entering the sleep state;
the processor comparing the sensor data stored in the buffer and the current sensor data with fall conditions set by a fall detection program; and
generating an alarm for a fall event if the sensor data meets the fall conditions; otherwise, the processor entering the sleep state.

8. The method as recited in claim 7, wherein the apparatus is a portable device worn on the person under care, the processor is a micro-processor of the apparatus, and the sensor unit is the accelerometer for generating the acceleration data correlated to the apparatus, or the accelerometer combined with other sensors such as gyroscope for generating the angular velocities.

9. The method as recited in claim 8, wherein the accelerometer is a three-axis accelerometer that is used to measure three acceleration component values in three axial directions, and the acceleration value is calculated according to the three acceleration components values.

10. (canceled)

11. The method as recited in claim 7, wherein the processor is set, by a power management unit, to be in the sleep state or to be woken up to be in an awake state for performing the method for human fall detection.

12. The method as recited in claim 7, wherein the buffer storing the sensor data is a buffer inside the processor, a buffer inside the sensor unit, or an external memory.

13. An apparatus for human fall detection, worn on a person under care, comprising:
a processor;
a power management unit inside the processor, used to set the processor to an awake state or a sleep state;
a sensor unit, being an accelerometer or an accelerometer combined with other sensors such as gyroscope operatively coupled with the processor, used to generate sensor data correlated to the apparatus; and
a memory unit, operatively coupled with the processor, used to store a fall detection program that is executed by the processor for performing a method for human fall detection comprising:
setting the processor in a sleep state;
storing the sensor data in a buffer, wherein the sensor data is continuously generated and the buffer is configured to store the latest limited sensor data;
waking the processor up from the sleep state when receiving a collision signal from the sensor unit, wherein the collision signal is generated by the accelerometer when the accelerometer determines that an acceleration value calculated from current sensor data is larger than a first threshold;
the processor comparing current acceleration value calculated from the current sensor data with a second threshold;
the processor retrieving the sensor data stored in the buffer and the current sensor data from the sensor unit if the current acceleration value is larger than the second threshold; otherwise, the processor entering the sleep state;
the processor comparing the sensor data stored in the buffer and the current sensor data with fall conditions set by a fall detection program; and
generating an alarm for a fall event if the sensor data meets the fall conditions; otherwise, the processor entering the sleep state.

14. The apparatus as recited in claim 13, wherein the apparatus is a portable device worn on the person under care, and the processor is a micro-processor of the apparatus.

15. The apparatus as recited in claim 13, wherein the accelerometer is a three-axis accelerometer that is used to measure three acceleration component values in three axial directions, and the acceleration value is calculated according to the three acceleration components values.

16. The apparatus as recited in claim 13, wherein the buffer storing the sensor data is a buffer inside the processor, a buffer inside the sensor unit, or an external memory.

17. The apparatus as recited in claim 13, wherein the sensor data stored in the buffer is the latest previous sensor data as compared with the generated current sensor data.

18. (canceled)

19. The apparatus as recited in claim 13, further comprising:
a communication unit operatively coupled with the processor, used to transmit an alarm to a care system when detecting the fall event.
